(19) United States
(12) Patent Application Publication
(10) Pub. No.: US 2008/0171706 A1
(43) Pub. Date: Jul. 17, 2008

(54) Anti-Adhesive Compounds to Prevent and Treat Bacterial Infections

(75) Inventors: Jenny Berglund, Oslo (NO); Julie Bouckaert, Sint-Genesius-Rode (BE); Henri De Greve, Brussel (BE); Stefan Knight, Uppsala (SE)

Correspondence Address: TRASK BRITT, P.O. BOX 2550, SALT LAKE CITY, UT, 84110, US

(73) Assignee: Vlaams Interuniversitair Instituut Voor Biotechnologie VZW, Brussel (BE)

(21) Appl. No.: 10/594,225

(22) PCT Filed: Mar. 23, 2005
(86) PCT No.: PCT/EP2005/051364

§ 371 Date: Oct. 5, 2007

(30) Foreign Application Priority Data
Mar. 23, 2004 (EP) 04101199.0

(51) Int. Cl. A61K 31/7004; A61P 31/04

(52) U.S. Cl. 514/23

(57) ABSTRACT

The present invention provides compounds and compositions capable of inhibiting the attachment of Gram-negative bacteria on a host epithelium. Accordingly, said compounds and compositions can for example be used for the manufacture of a medicament to treat urinary, lung and gastrointestinal infections caused by said Gram-negative bacteria

### FIELD OF THE INVENTION

The present invention provides compounds and compositions capable of inhibiting the attachment of Gram-negative bacteria on a host epithelium. Accordingly, said compounds and compositions can for example be used for the manufacture of a medicament to treat urinary, lung and gastrointestinal infections caused by said Gram-negative bacteria.

### BACKGROUND OF THE INVENTION

Many pathogenic Gram-negative bacteria such as Escherichia coli, Proteus species, Haemophilus influenzae, Salmonella enteriditis, Salmonella typhimurium, Bordetella pertussis, Yersinia enterocolitica, Helicobacter pylori and Klebsiella pneumoniae assemble hair-like adhesive organelles called pill on their surfaces. Pili frequently mediate microbial attachment, often the essential first step in the development of disease, by binding to receptors present in host tissues and may also participate in bacterial-bacterial interactions important in biofilm formation. For example uropathogenic strains of E. coli (UPEC) possess pili that bind to receptors present on uroepithelial cells, causing urinary tract infection (UTI). UTI is one of the most common bacterial infections, estimated to affect at least 50% of women over life at a yearly cost of ˜$2 billion in the US alone (Foxman, 2002). The most common cause of UTI is infection by UPEC, which accounts for about 80% of reported cases (Ronald, 2002). Most UTIs can be effectively treated with antibiotics, but recurrence is a problem as is the emergence of antibiotic resistant strains (Ronald, 2002; Gupta et al., 2001; Nicolle, 2002; Johnson et al., 2002). For attachment to the uroepithelium, UPEC express a number of carbohydrate binding adhesins (Mulvey, 2002; Schilling et al., 2001; Berglund and Knight, 2003). These adhesins mediate specific binding to carbohydrate-containing receptors in the uroepithelium, thereby determining the tissue tropism of the bacteria. The differential expression of cell surface receptors in different parts of the urinary tract allows UPEC expressing different adhesins to generate very different clinical outcomes. For example, P-piliated UPEC cause pyelonephrities by binding to galabiose-containing receptors in the kidney epithelium. Mannose-binding type-1 pili promote infection of the bladder epithelium (cystitis) by targeting uroplakin high-mannose receptors present on the surface of the superficial umbrella cells lining the mucosal surface of the urinary bladder. Of the various UPEC adhesins, type-1 pill are by far the most abundant (Brinton, 1959; Buchanan et al., 1985; O'Hanley et al., 1985; Langermann et al., 1997; Bahrani-Mougeot et al., 2002). Type-1 pili consist of a cylindrical rod of repeating immunoglobulin-like (Ig-like) FimA subunits, followed by a short and stubby tip fibrillum. These structures are assembled by the chaperonelusher pathway (Thanassi et al., 1998; Knight et al, 2000; Sauer et al., 2000 a; Sauer et al., 2000 b) and in their mature form the Ig fold of every constituent subunit is completed by an amino-terminal extension from a neighboring subunit in a process termed ‘donor strand exchange’ (Choudhury et al., 1999; Sauer et al., 1999; Zavialov et al., 2003). FimH is a two-domain adhesin protein at the end of the tip fibrillum, responsible for the mannose-sensitive bacterial adhesion. The amino-terminal lectin domain (residues 1-158) is joined to a carboxy-terminal pilin domain (residues 159-279) that links the adhesin to the rest of the pilus. The primary physiological receptor for FimH in the urinary tract is the glycoprotein uroplakin 1a (Zhou et al., 2001), but FimH recognizes a wide range of glycoproteins carrying one or more N-linked high-mannose structures. FimH also binds yeast mannans and mediates agglutination of yeast cells. FimH alleles from different E coli isolates are highly conserved (Hung et al., 2002). Nevertheless, minor sequence differences have been shown to correlate with different binding and adhesion phenotypes (Sokurenko et al., 1994; Sokurenko et al., 1995; Sokurenko et al., 1997; Sokurenko et al., 1998). Most UPEC strains carry FimH variants that allow tight binding to substrates with a terminal alpha-linked D-mannose (e.g. mannosylated bovine serum albumin or yeast mannans), whereas the majority of fecal strains carry FimH variants that require trimannosides for tight binding (Sokurenko et al, 1995; Sokurenko et al., 1997). It is known in the art that FimH-mediated adhesion can be inhibited by D-mannose and also by a variety of natural and synthetic saccharides containing terminal mannose residues (WO0110386 and (Firon et al., 1982; Firon et al., 1983; Firon et al., 1984; Lindhorst et al., 1998; Neeser et al., 1986). Indeed blocking of the FimH-receptor interaction has been shown to prevent bacterial adhesion to bladder uroepithelium and infection (Langermann et al., 1997; Thankavel et al., 1997; Langermann et al., 2000). However, there is a need for molecules with superior binding affinities—with FimH—which have at the same time favourable in vivo effects. In the present invention we have developed a simple and reliable assay for measuring ligand binding to FimH and have used this assay to determine dissociation constants for a variety of chemically synthesized alpha-D-mannose derivatives (several alkyl and aromatically substituted mannosides). We show that several of these molecules have nanomolar activities with FimH. Thus the present invention provides new molecules which can be used for the inhibition of binding of type-1 pili with host tissue and hence said molecules can be used for the manufacture of medicines to treat bacterial infections caused by Gram-negative bacteria possessing type-1 pili.

### FIGURES AND TABLES

FIG. 1. (A) Binding curve of α-D-mannose. (B) Displacement curve of butyl mannoside. (C) Linear dependency of ΔG⁰ for FimHtr_{J96} binding on number of methyl groups in alkyl mannosides with up to 8 methyl groups in the alkyl chain.

FIG. 2. (A) Binding profiles for three different FimH variants from strains J96, F18 and Cl#4 to a series of trimannoses. All three strains follow the same binding trend although J96 binding is stronger to all compounds. (B) The tri-mannosides correspond to the branches of the high-mannose tree (left). α1-3, α1-6 mannopentaose is the oligomannose on the right hand side.

Table 1: K_{D} and calculated ΔG⁰ for a series of O1 alkyl and aryl mannosides. The Surface Plasmon Resonance and Displacement binding experiments define heptyl α-D-mannopyranoside as the best binder

Table 2: K_{D} and calculated ΔG⁰ for mono- and disaccharides and a deoxy-mannose. Other mono- and disaccharides and a deoxy mannose do not reach the high affinity of the mannose for FimH. Fructose, present at a concentration of ≈5% in fruit juices, follows mannose with an only 15 times lower affinity, as reported earlier by Zafriri et al. 1989.

Table 3. K_{D} and calculated ΔG⁰ for a series of tri-mannosides (FIG. 3) binding to FimH from three different strains. (nd=not determined)

### DETAILED DESCRIPTION OF THE INVENTION

Unless defined otherwise, all technical and scientific terms used herein have the same meaning as is commonly understood by one of skill in the art to which the invention belongs. All patents, patent applications, published applications and publications, Genbank sequences, websites and other published materials referred to throughout the entire disclosure herein, unless noted otherwise, are incorporated by reference in their entirety. In the event that there is a plurality of definitions for terms herein, those in this section prevail. Where reference is made to an URL or other such identifier or address, it is understood that such identifiers can change and particular information on the Internet can come and go, but equivalent information is known and can be readily accessed, such as by searching the internet and/or appropriate databases. Reference thereto evidences the availability and public dissemination of such information.

In the present invention, the inventors have designed and fabricated compounds which interfere with the adhesion of Gram-negative bacteria to mannose oligosaccharides located on the host epithelium thereby reducing the capacity of said piliated bacteria to attach to and infect host tissues. In a particular embodiment said Gram-negative bacteria comprise type-1 pill. More specifically the compounds of the present invention interfere with the binding of FimH and homologues thereof with mannose oligosaccharides present on a host epithelial tissue. The compounds of the present invention are alpha-D-mannose derivatives (also designated in the art as alpha-D-mannopyranoside-derivatives) which are useful in treating bacterial diseases caused by Gram-negative bacteria. Additionally the compounds can also be used in preventing costly biofilm formation in medical, industrial and various other settings.

Thus in a first embodiment the invention provides the use of

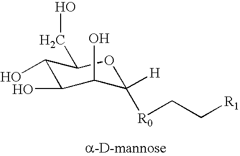

wherein R₀═O, CH₂ or S
and
—R₁═—CH₂CH₃ (ethyl), or
   —CH₂CH₂CH₃ (n-propyl), or
   —CH₂CH₂CH₂COOH (₃ carboxypropyl), or
   —CH₂CH₂CH₂CHO (4-oxobutyl), or
   —CH₂CH₂CH₂CH₃ (n-butyl), or
   —CH₂CH₂CH₂CF₃ (4,4,4-trifluorobutyl), or
   —CH₂CH₂CH₂CH₂OH (4-hydroxybutyl), or
   —CH₂CH₂CH₂CH₂CHO (5-oxopentyl), or
   —CH₂CH₂CH₂CH₂CH₃ (n-pentyl), or
   —CH₂CH₂CH₂CH₂CF₃ (5,5,5-trifluoropentyl), or
   —CH₂CH₂CH₂CH₂COOH (4-carboxybutyl), or
   —CH₂CH₂CH₂CH₂NH₂ (4-aminobutyl), or
   —C₆H₁₁OH (4-hydroxycyclohexyl), or
   —C₆H₁₁CF₃ (4-trifluoromethylcyclohexyl), or
   —C₆H₅ (phenyl), or
   —C₆H₄OH (p-hydroxyphenyl), or
   —C₆H₄NH₂ (p-aminophenyl), or
   —C₆H₄NO₂ (p-nitrophenyl), or
   —C₆H₄COOH (p-carboxyphenyl), or
   —C₆H₄CH₃ (p-methylphenyl), or
   —C₆H₄CF₃ (p-trifluoromethylphenyl), or
   —C₆H₄CHO (p-formylphenyl), or
   —C₄H₅N₂ (pyrimidyl), or
   —C₄H₄N₂OH (2-hydroxypyrimidyl), or
   —C₆H₁₁ (cyclohexyl)
      for the manufacture of a medicament to treat a subject suffering from infection of a Gram-negative bacterium. In a particular embodiment said Gram-negative bacterium possesses a type-1 adhesion.

In a particular embodiment the molecules used in the invention (as defined by claim 1) are homodimers. In another particular embodiment said compounds are homotrimers. In yet another particular embodiment said compounds are homotetramers.

In a non-limiting example the synthesis of homodimers (also defined herein as branched mannoside compounds) is presented in example 6.

As utilized herein, the term “pilus” or “pili” relates to fibrillar, heteropolymeric structures embedded in the cell envelope of many tissue-adhering pathogenic bacteria, notably pathogenic Gram-negative bacteria. In the present specification, the terms pilus and pili are used interchangeably. A pilus is composed of a number of “pilus subunits” which constitute distinct functional parts of the intact pilus.

The phrase “preventing or inhibiting binding between pilus and a host epithelial tissue” indicates that the normal interaction between a type-1 pilus and its natural ligand on the epithelial tissue is being affected either by being inhibited, or reduced to such an extent that the binding of the pilus to the host epithelial tissue is measurably lower than is the case when the pilus is interacting with the host epithelial tissue at conditions which are substantially identical (with regard to pH, concentration of ions, and other molecules) to the native conditions in the environment (e.g. the bladder, the kidney, the intestine, the lung). Measurement of the degree of binding can be determined in vitro by methods known to the person skilled in the art (microcalorimetry, radioimmunoassays, enzyme based immunoassays, fluorescent labeling of the bacteria etc.).

The compounds and compositions of the present invention which prevent or inhibit binding between type-1 pilus and epithelial tissue are said to exhibit “antibacterial activity.” By the term “host” is in the present context meant a host (or subject), which can be any plant or animal, including a human being, who is infected with, or is likely to be infected with, tissue-adhering pilus-forming bacteria which are believed to be pathogenic. By the term “an effective amount” is meant an amount of the compound in question which will in a majority of hosts (e.g. patients) have either the effect that the disease caused by the pathogenic bacteria is cured or ameliorated or, if the substance has been given prophylactically, the effect that the disease is prevented from manifesting itself. The term “an effective amount” also implies that the substance is given in an amount which only causes mild or no adverse effects in the subject to whom it has been administered, or that the adverse effects may be tolerated from a medical and pharmaceutical point of view In the light of the severity of the disease for which the substance has been given.

As used herein “treatment” includes both prophylaxis and therapy. Thus, in treating a subject, the compounds of the invention may be administered to a subject already harboring a bacterial infection or in order to prevent such infection from occurring or to prevent infection from re-occurring. For example in the case of urinary tract infections it is important to realize that these infections are often recurrent (20-25% in women). The current treatment is a prophylactic treatment with antibiotics for up to six months. In the case of reflux (of urine to kidneys) in newborn babies, prophylactic treatment is advised for over one year to prevent kidney disfunctionality. The molecules of the present invention can be a valuable alternative for prophylactic treatments with antibiotics. In another alternative the molecules of the invention can be administered together with antibiotics.

In yet another embodiment the molecules of the invention can be used for the manufacture of a medicament to treat bacterial infections caused by bacteria selected from the list consisting of Klebsiella pneumoniae, Haemophilus influenza, Shigella species, Salmonella typhimurium, Bordetella pertussis, Yersinia enterolytica, Helicobacter pylor, Proteus species and Escherichia coli.

Some examples of diseases caused by these pathogenic Gram-negative bacteria are gastroenteritis (E. coli, Salmonella, Shigella and Yersinia), urinary tract infections (E. coli), dysentery (Shigella and Escherichia coli), pneumonia (Klebsiella). All these diseases can be treated by the molecules of the present invention.

In another embodiment the compounds of the invention (which are antibacterial compositions) may be utilized to inhibit pili adhesion by providing an effective amount of such compositions to a host (e.g. patient).

In particularly for use as antimicrobials for the treatment of animal subjects, the compounds of the invention can be formulated as pharmaceutical or veterinary compositions. Depending on the subject to be treated, the mode of administration, and the type of treatment desired, e. g., prevention, prophylaxis, therapy; the compounds are formulated in ways consonant with these parameters. A summary of such techniques is found in Remington's Pharmaceutical Sciences, latest edition, Mack Publishing Co., Easton, Pa.

The term ‘medicament to treat’ relates to a composition comprising molecules as described herein above and a pharmaceutically acceptable carrier or excipient (both terms can be used interchangeably) to treat or to prevent diseases as described herein. The administration of a molecule or a pharmaceutically acceptable salt thereof may be by way of oral, inhaled, topical or parenteral administration. The active compound may be administered alone or preferably formulated as a pharmaceutical composition. An amount effective to treat bacterial infections caused by Gram-negative bacteria depends on the usual factors such as the nature and severity of these infections being treated and the weight of the mammal. Doses will normally be administered continuously or once or more than once a day, for example 2, 3, or 4 times a day, more usually 1 to 3 times a day, such that the total daily dose is normally in the range of 0.0001 to 1 mg/kg; thus a suitable total daily dose for a 70 kg adult is 0.01 to 50 mg, for example 0.01 to 10 mg or more usually 0.05 to 10 mg. It is greatly preferred that the compound or a pharmaceutically acceptable salt thereof is administered in the form of a unit-dose composition, such as a unit dose oral, parenteral, topical or inhaled composition. Such compositions are prepared by admixture and are suitably adapted for oral, inhaled, topical or parenteral administration, and as such may be in the form of tablets, capsules, oral liquid preparations, powders, granules, ointments, lozenges, reconstitutable powders, injectable and infusable solutions or suspensions or suppositories or aerosols. Tablets and capsules for oral administration are usually presented in a unit dose, and contain conventional excipients such as binding agents, fillers, diluents, tabletting agents, lubricants, disintegrants, colourants, flavourings, and wetting agents. The tablets may be coated according to well known methods in the art. Suitable fillers for use include cellulose, mannitol, lactose and other similar agents. Suitable disintegrants include starch, polyvinylpyrrolidone and starch derivatives such as sodium starch glycollate. Suitable lubricants include, for example, magnesium stearate. Suitable pharmaceutically acceptable wetting agents include sodium lauryl sulphate. These solid oral compositions may be prepared by conventional methods of blending, filling, tabletting or the like. Repeated blending operations may be used to distribute the active agent throughout those compositions employing large quantities of fillers. Such operations are, of course, conventional in the art. Oral liquid preparations may be in the form of, for example, aqueous or oily suspensions, solutions, emulsions, syrups, or elixirs, or may be presented as a dry product for reconstitution with water or other suitable vehicle before use. Such liquid preparations may contain conventional additives such as suspending agents, for example sorbitol, syrup, methyl cellulose, gelatin, hydroxyethylcellulose, carboxymethyl cellulose, aluminium stearate gel or hydrogenated edible fats, emulsifying agents, for example lecithin, sorbitan monooleate, or acadia; non-aqueous vehicles (which may include edible oils), for example, almond oil, fractionated coconut oil, oily esters such as esters of glycerine, propylene glycol, or ethyl alcohol; preservatives, for example methyl or propyl p-hydroxybenzoate or sorbic acid, and if desired conventional flavoring or coloring agents. Oral formulations also include conventional sustained release formulations, such as tablets or granules having an enteric coating. Preferably, compositions for inhalation are presented for administration to the respiratory tract as a snuff or an aerosol or solution for a nebulizer, or as a microfine powder for insufflation, alone or in combination with an inert carrier such as lactose. In such a case the particles of active compound suitably have diameters of less than 50 microns, preferably less than 10 microns, for example between 1 and 5 microns, such as between 2 and 5 microns. A favored inhaled dose will be in the range of 0.05 to 2 mg, for example 0.05 to 0.5 mg, 0.1 to 1 mg or 0.5 to 2 mg. For parenteral administration, fluid unit dose forms are prepared containing a compound of the present invention and a sterile vehicle. The active compound, depending on the vehicle and the concentration, can be either suspended or dissolved. Parenteral solutions are normally prepared by dissolving the compound in a vehicle and filter sterilising before filling into a suitable vial or ampoule and sealing. Advantageously, adjuvants such as a local anaesthetic, preservatives and buffering agents are also dissolved in the vehicle. To enhance the stability, the composition can be frozen after filling into the vial and the water removed under vacuum. Parenteral suspensions are prepared in substantially the same manner except that the compound is suspended in the vehicle instead of being dissolved and sterilised by exposure to ethylene oxide before suspending in the sterile vehicle. Advantageously, a surfactant or wetting agent is included in the composition to facilitate uniform distribution of the active compound. Where appropriate, small amounts of bronchodilators for example sympathomimetic amines such as isoprenaline, isoetharine, salbutamol, phenylephrine and ephedrine; xanthine derivatives such as theophylline and aminophylline and corticosteroids such as prednisolone and adrenal stimulants such as ACTH may be included. As is common practice, the compositions will usually be accompanied by written or printed directions for use in the medical treatment concerned. The present invention further provides a pharmaceutical composition for use in the treatment and/or prophylaxis of herein described bacterial infections which comprises a pharmaceutically acceptable salt thereof, or a pharmaceutically acceptable solvate thereof, and, if required, a pharmaceutically acceptable carrier thereof. In a particular embodiment the molecules of the invention can be used for the manufacture of a medicament to treat a urinary infection. In a more particular embodiment said urinary infection is caused by E. coli.

In yet another embodiment the molecules of the invention can be used for the manufacture of a medicament to treat a gastrointestinal infection. In a more particular embodiment said gastrointestinal infection is caused by Escherichia, Salmonella, Shigella or Yersinia species.

It will be understood that the appropriate dosage of the molecules should suitably be assessed by performing animal model tests, wherein the effective dose level and the toxic dose level as well as the lethal dose level are established in suitable and acceptable animal models. Further, if a substance has proven efficient in such animal tests, controlled clinical trials should be performed. Needless to state such clinical trials should be performed according to the standards of Good Clinical Practice.

In a particular embodiment the compounds of the invention can be used alone or in combination with other antibiotics such as erythromycin, tetracycline, macrolides, for example azithromycin and the cephalosporins. Depending on the mode of administration, the compounds will be formulated into suitable compositions to permit facile delivery to the affected areas.

Formulations may be prepared in a manner suitable for systemic administration or topical or local administration. Systemic formulations include those designed for injection (e. g., intramuscular, intravenous or subcutaneous injection) or may be prepared for transdermal, transmucosal, or oral administration. The formulation will generally include a diluent as well as, in some cases, adjuvants, buffers, preservatives and the like.

In a particular embodiment the antibacterial compositions of the present invention have a variety of industrial uses, well known to those skilled in such arts, relating to their antibacterial properties. In general, these uses are carried out by bringing a biocidal or bacterial inhibitory amount of the antibacterial compositions of the present invention into contact with a surface, environment or biozone containing Gram-negative bacteria so that the composition is able to interact with and thereby interfere with the biological function of such bacteria. For example, such antibacterial compositions can be used to prevent or inhibit biofilm formation caused by Gram-negative bacteria and to inhibit bacterial colonization by a Gram-negative organism. Compositions may be formulated as sprays, solutions, pellets, powders and in other forms of administration well known to those skilled in such arts.

It should be understood that compounds of the present invention may be used as lead compounds in pharmaceutical efforts to synthesize variants that can be used for the treatment of several types of disease caused by pathogenic Gram-negative bacteria such as Escherichia coli, Haemophilus influenzae, Salmonella enteriditis, Salmonella typhimurium, Bordetella pertussis, Yersinia enterocolitica, Helicobacter pylori, Proteus species and Klebsiella pneumoniae.

The present invention is further illustrated by the following examples which should not be construed as limiting in any way. The contents of all cited references (including literature references, issued patents, published patent applications, and co-pending patent applications) cited throughout this application are hereby expressly incorporated by reference.

### EXAMPLES

1. Binding Studies Between FimH and Mannose Derivatives

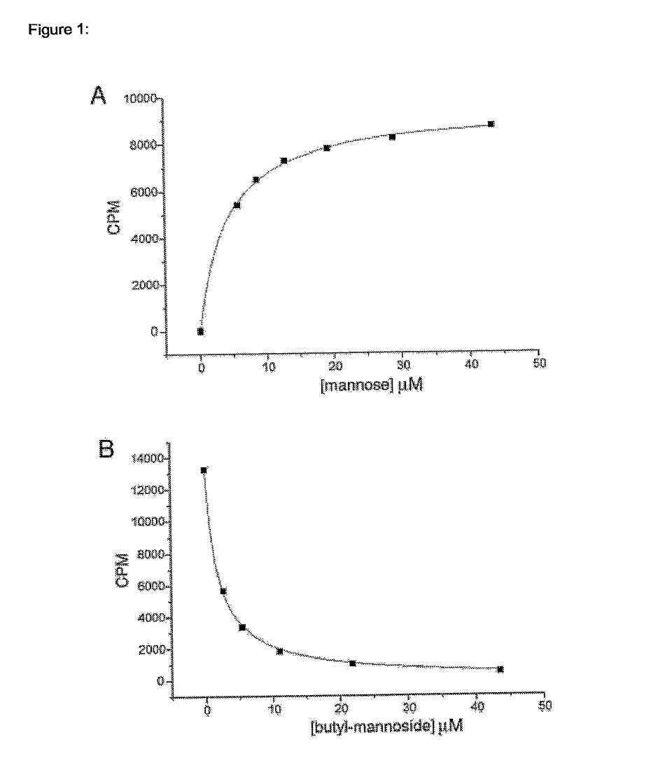

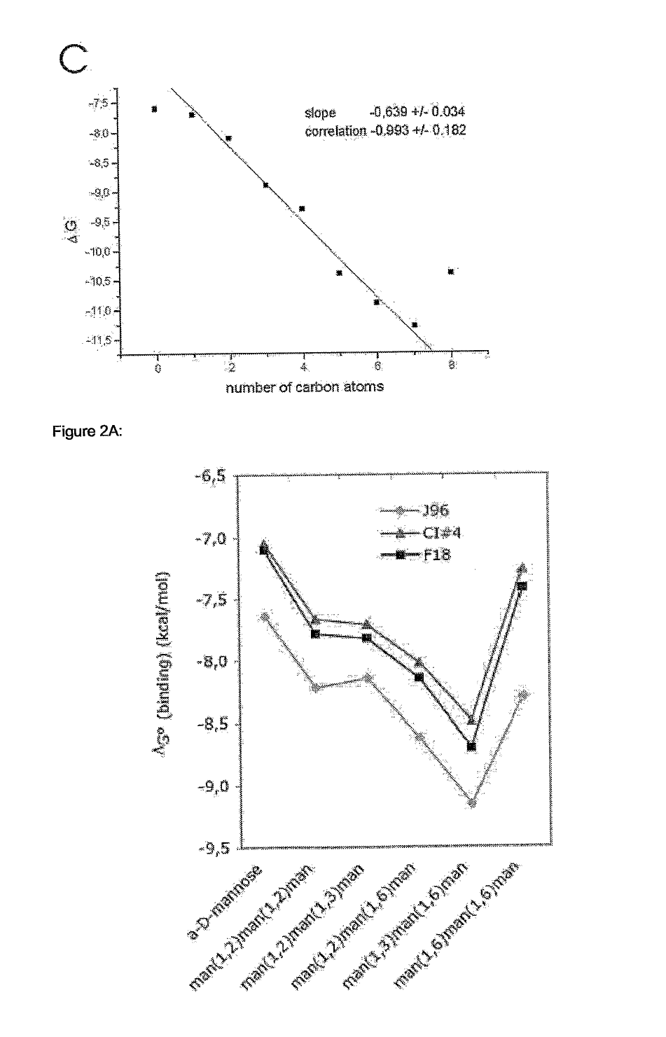

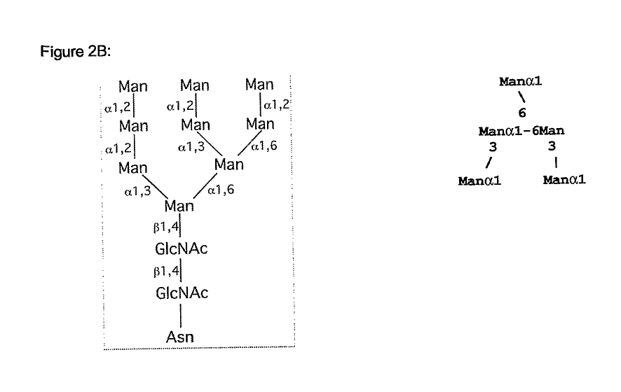

To measure the dissociation constant for FimH:alkyl or aryl-mannoside binding, two different binding assays were developed. The first binding assay uses [³H]-mannose. The amount of radioactively labeled mannose bound to FimHtr_{J96} was measured at six different concentrations of [³H]-mannose, and a hyperbolic curve fitted to the resulting data (FIG. 1A). FimHtr_{J96} corresponds to the carbohydrate (mannose) binding domain of FimH from the uropathogenic E. coli strain J96. The dissociation equilibrium constant was determined from this graph at the concentration of mannose halfway to equilibrium, which corresponds to occupation of half of the binding sites.

Surface Plasmon Resonance measurements were performed on a Biacore3000™. In a first experiment, the kinetic constants and maximal binding were determined for the FimH-antibody interaction. Next, a fixed concentration of FimH (close to the K_{D} of the FimH-antibody interaction) in combination with varying concentrations of carbohydrate, were used to determine the dissociation equilibrium constant of the FimH-saccharide interaction in a competition experiment. Every measurement was repeated at least twice, including testing the variation between different protein and saccharide batches.

To eliminate the possibility of different binding strength for full length FimH and FimHtr_{J96}, the binding of alpha-D-mannose to FimC:FimH complex was also measured. A value of K_{D}=2.3 μM was obtained, in good agreement with the value measured using FimHtr_{J96}. The inhibition of [³H]-mannose binding was used in a displacement assay to determine the dissociation constant for a synthetic butyl mannoside by measuring the amount of [³H]-mannose bound to the protein in the presence of increasing amounts of the inhibitor (FIG. 1B). A dissociation constant of K_{D}=0.15 μM for butyl mannoside was determined using this procedure, around 15 times stronger than for D-mannose. To investigate the effect of sequential addition of methyl groups to the O1 oxygen of D-mannose, a series of alkyl mannosides were synthesized and the dissociation constants determined using the [³H]-mannose displacement assay and by surface plasmon resonance (Table 1).

There is a near-linear correlation between the binding free energy as determined from the measured dissociation constants and the number of methyl groups in the alkyl mannoside, with each additional methyl group contributing about −0.4 kcal mol⁻¹ of binding energy (FIG. 1C). Aromatically substituted mannosides have been reported to be particularly potent inhibitors of FimH-mediated bacterial adhesion (Firon et al., 1987). Using our displacement assay, the dissociation constants were measured for four such compounds, ethylphenyl alpha-D-man, ethyl aminophenyl alpha-D-man, pNP alpha-D-man and MeUmb alpha-D-man. In concordance with the earlier results, those compounds bind very tightly to FimHtr_{J96} (K_{D}=86 nM for ethylphenyl alpha-D-man, K_{D}=137 nM for ethyl aminophenyl alpha-D-man, K_{D}=26 nM for pNP alpha-D-man, K_{D}=12 nM for MeUmb alpha-D-man).

2. Binding of Mono- and Trimannosides to Fecal and UPEC FimH Variants

FimH alleles from different E. coli isolates exhibit only minor sequence differences, but nevertheless mediate significant variations in adhesion properties (Sokurenko et al., 1994; Sokurenko et al., 1995; Sokurenko et al., 1997; Sokurenko et al., 1998). To investigate if these variations reflect differences in sugar binding at the molecular level, the dissociation constants of a series of mannosides for the FimH lectin domain from a fecal (F18) and from a UPEC (Cl#4) strain were determined. These two FimH variants have previously been shown to mediate significantly different adhesion patterns (Sokurenko et al., 1995). For comparison, binding to FimHtr_{J96} was also investigated. Both F18 and Cl#4 FimH lectin domains were cloned, expressed, and purified in the same way as FimHtr_{J96} (Schembri et al., 2000). These two lectin domains differ from the lectin domain of FimH_{J96} by substitutions Val27Ala, Asn70Ser and Ser78Asn. In addition, FimH_{Cl#4} differs from the other two variants by a Gly73Glu substitution. None of these residues are close to the mannose binding pocket. Five different tri-mannosides corresponding to high-mannose substructures were synthesized, and their binding to FimHtr_{J96}, FimHtr_{F18} and FimHtr_{Cl#4} measured using our [³H]-mannose displacement assay (Table 3). Alpha-D-mannose binding was first directly measured for each of the FimH variants. The measured dissociation constants for mannose binding to FimHtr_{F18} (K_{D}=10 μM) and to FimHtr_{Cl#4} (K_{D}=11 μM) are virtually identical, approximately four-fold higher than for FimHtr_{J96} (K_{D}=2.3 μM). Tri-saccharide affinities lie in the range K_{D}=0.5-7.5 μM. A similar trisaccharide binding pattern is observed for all FimH variants studied, but the J96 variant binds approximately two-fold tighter than the F18 and Cl#4 variants to all of the tri-saccharides (FIG. 3). Pentamannose binds with the highest affinity to FimHtr_{J96} (K_{D}=330 nM).

3. Synthesis of Alkyl-Mannoside Compounds

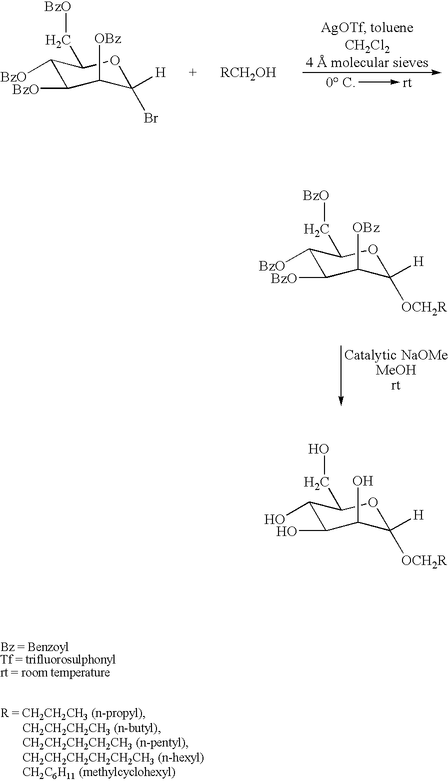

4. Synthesis of Alkylthiomannoside Compounds

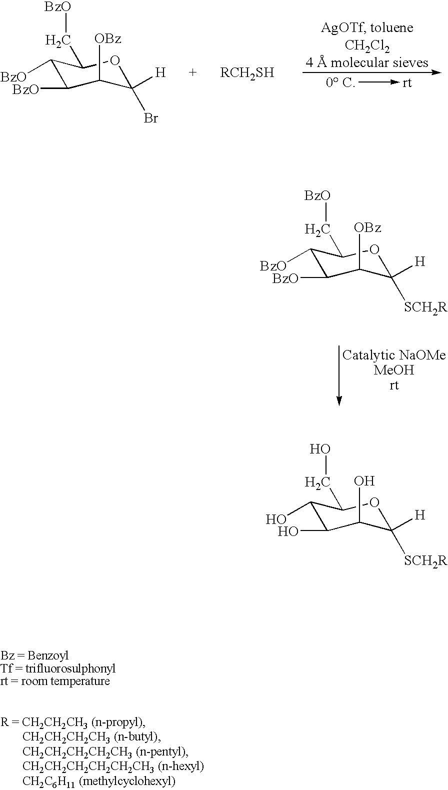

5. Synthesis of C-glycosyl Compounds

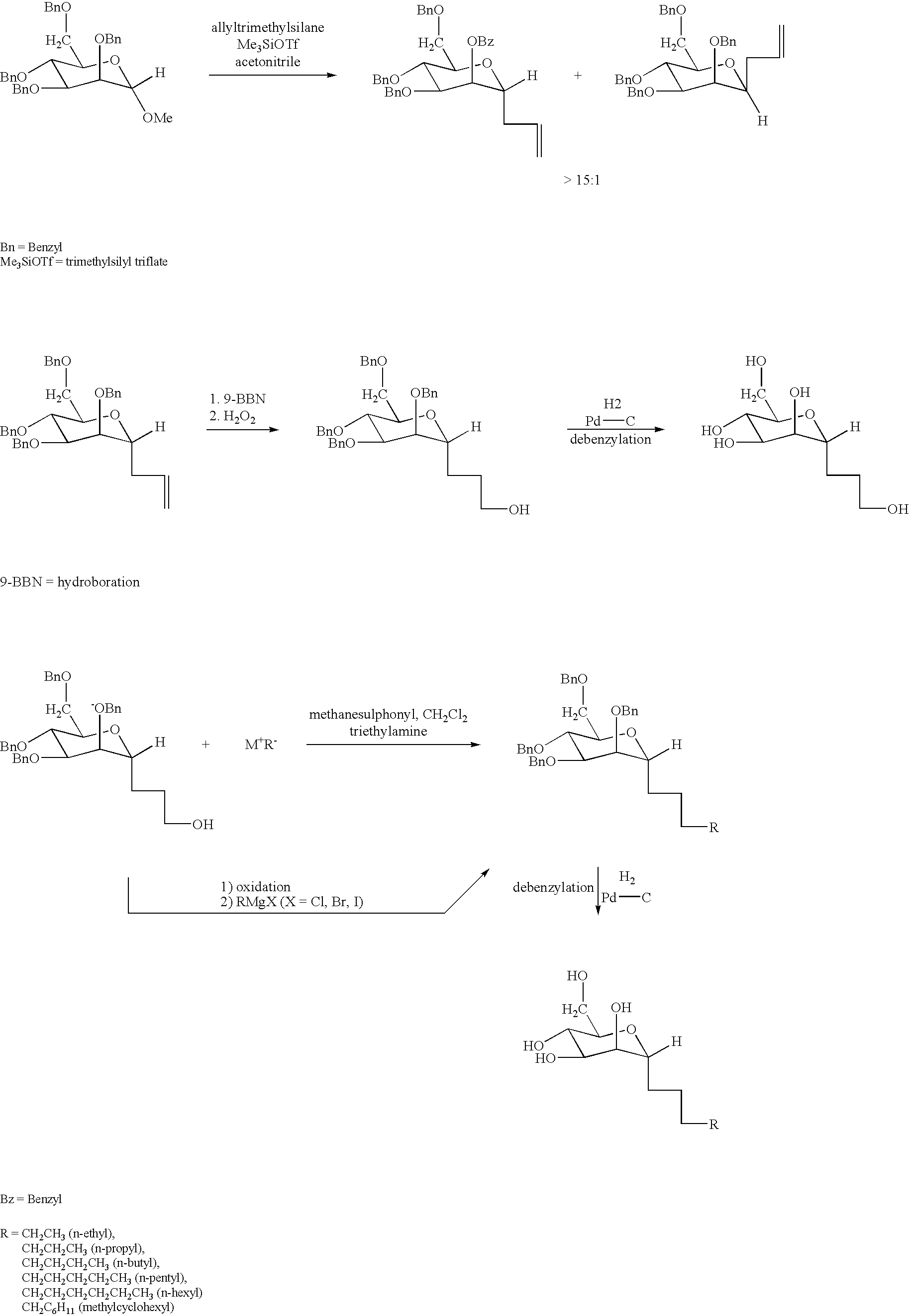

6. Synthesis of Branched Mannoside Compounds

Because of its unique properties, high reactivity, stability to air, and remarkable functional group tolerance, benzylidenebis(tricyclohexylphosphine)dichlororuthenium 4 (Grubbs' catalyst) (Nguyen et al., 1993) is an excellent catalyst for the synthesis of homodimers from O-alkenylmannopyranosides 3. The latter compounds can be prepared by a silver promoted reaction of tetrabenzoylated (or eventually tetra acetylated-) mannopyranosyl bromide 1 with terminal alken-1-ols 2. Using commercially available alkenols, the chain length between both sugar units may vary between 6, 8, 10 or 12 carbon atoms. A precedent of such a homodimerisation reaction has been described in literature (Dominique et al., 1998). The product 5, after the metathesis reaction will be obtained as a mixture of E and Z diastereomers. After Zemplen deprotection and catalytic hydrogenation of the double bond the final product 7 will be obtained.

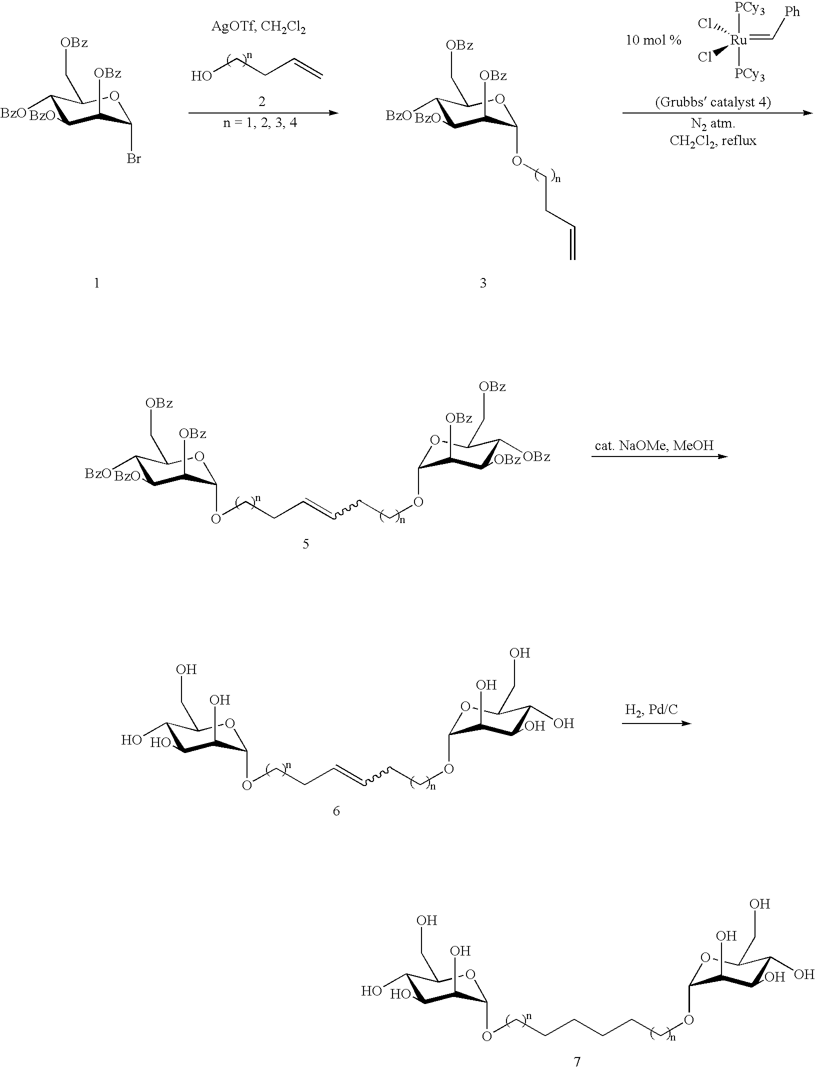

In order to synthesise also the dimers connected by an odd number of carbon atoms, a Sonogashira cross coupling reaction may be executed between ω-iodoalkenyl mannopyranoside 8 and the alkynyl mannopyranoside 9. This way, the chain length between both sugar units may vary between 8-14 C-atoms (also odd numbers). Compounds 10 and 12 are conformationally restricted and most likely will be formed as a mixture of diastereomers. Complete reduction of the unsaturated bonds leads finally to compound 11

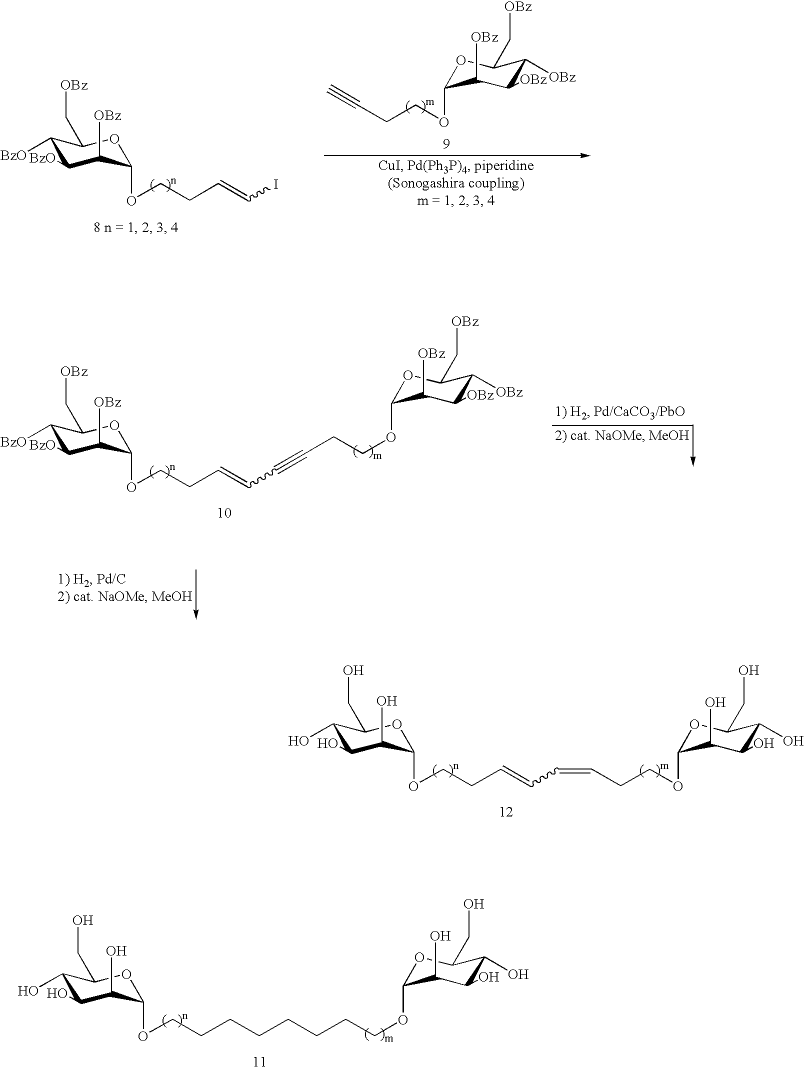

7. Ex vivo Testing of Synthetic Mannose Derivatives

7.1. Yeast Agglutination Assay

The binding of type 1 positive bacteria is assayed by their ability to agglutinate yeast cells (Saccharomyces cerevisiae) on glass slides. Aliquots of washed bacterial suspensions at OD₆₆₀ 0.5% and 5% yeast cells are mixed and the time until agglutination occurres is measured. Mannoside derivatives are added to evaluate their influence on agglutination of yeast cells.

Alternatively, binding to yeast cells is assayed by incubating aliquots of bacteria with yeast cells for 2 min. After removal of unbound bacteria, mannoside derivatives are added to release the attached bacteria from the yeast cells. The bacteria are then quantified by plating out.

7.2. Adhesion Inhibition Assays

Inhibitor titration of bacterial binding to mannan bound to 96-well plates with mannoside derivatives is carried out as described (Sokurenko et al., 1995; Sokurenko et al., 1997; Knudsen and Klemm, 1998).

We use the fim-null mutant AAEC185 strain (Blomfield et al., 1991), transformed with the plasmids pUT2002 (Minion et al., 1989) and pMMB66 (Furste et al., 1986). The plasmid pUT2002 carries the fim operon with a deletion in the fimH gene encoding the FimH adhesin. The plasmid pMMB66 is a low-copy number plasmid with the lad repressor and the tac promoter, controlling the expression of the cloned wild-type fimH gene. The strain AAEC185 (pUT2002) produces morphologically and antigenically indistinguishable type 1 fimbriae that are nonadherent The strain AAEC185 (pUT2002) (pMMB66) produces FimH positive type 1 piliated bacteria after induction. Both strains AAEC185 (pUT2002) and AAEC185 (pUT2002) (pMMB66) are grown overnight at 37° C. in a shaking incubator. The next day the bacteria are diluted 10 times and further grown overnight at 37° C. without shaking in the presence of 1 μM IPTG. The presence of IPTG results in an optimal expression of the fimH gene carried by the pMMB66 plasmid and the production of wild type adhering type 1 fimbriae. The density of bacteria used in all assays is 10⁷ colony forming units per 100 μl.

7.3. Quantitative adhesion assay: Wells are coated with mannan (Sigma) at a concentration of 10 μg/ml, washed three times with PBS and subsequently coated with 0.2% bovine serum albumin (BSA) in PBS. Bacterial cell suspensions with identical cell numbers in PBS and 0.1% BSA are added and incubated for 40 min at 37° C. without shaking. The wells are then washed three times with PBS, and 160 μl LB containing 100 mM methyl α-D-mannose is added to each well and incubated for 5 h at 37° C. to remove the bound bacteria. The number of bound bacteria is determined by a growth assay as described (Sokurenko et al., 1995) or by measuring OD₆₀₀ values with a micro-titre plate reader (Knudsen and Klemm, 1998).

7.4. Quantitative inhibition assay: Wells are coated with mannan (Sigma) at a concentration of 10 μg/ml, washed three times with PBS and subsequently coated with 0.2% bovine serum albumin (BSA) in PBS. Bacterial cell suspensions with identical cell numbers in PBS containing 0.1% BSA are mixed with increasing concentrations of mannoside derivatives, added to the mannan-containing wells and incubated for 40 min at 37° C. without shaking. The wells are then washed with PBS, and 160 μl LB containing 100 mM methyl α-D-mannose is added to each well and incubated for 5 h at 37° C. to remove the bound bacteria. The number of bound bacteria is determined by a growth assay as described (Sokurenko et al., 1995) or by measuring OD₆₀₀ values with a micro-titre plate reader (Knudsen and Klemm, 1998).

7.5. Adhesion-Inhibition to Human Bladder Cell Line

The human carcinoma cell line 5637 (ATCC HTB-9) is derived from the urinary bladder. This cell line is propagated at 37° C. in RPMI 1640 tissue culture medium supplemented with 10% fetal bovine serum. The cell line is subcultured 2 to 3 times per week. The original medium is removed and the cells are rinsed with a solution of 0.25% trypsin and 0.03% EDTA. The rinse solution is removed and 1 to 2 ml of trypsin-EDTA solution is added. The flask is kept at room temperature (or at 37° C.) until the cells detach. Fresh culture medium is added, aspirated and dispensed into new culture flasks.

The bacterial strains AAEC185 (pUT2002) and AAEC185 (pUT2002) (pMMB66) are cultured as described above to express type 1 pili. The bacterial cells are harvested during the exponential phase, washed in and diluted in PBS till OD₆₀₀=1. For inhibition of the adhesion, the smallest concentration of the carbohydrate required to completely block adhesion is determined by adding serial dilutions of the inhibiting carbohydrate to the buffer. An estimation of the lowest concentration can be obtained from the quantitave inhibition assay described above. Adhesion and inhibition of adhesion of bacterial cells to the bladder cell line can be visualized with fluorescent labeled antibodies directed against the type 1 fimbriae (Falk et al., 1994) and examined microscopically.

7.6. In Silico Prediction of the Interaction Between Ligands and Macromolecules

To predict the interaction between the FimH adhesin and the synthesized ligands, the dissociation constants and the binding-interactions are calculated using structure-based computer-assisted dug-design, also called docking. Docking techniques allow translations, rotations and conformational flexibility of the inhibitor to search for the best possible binding orientation and conformation in the FimH binding site. A program called AutoDock3 (Morris et al., 1998) was developed for structure-assisted drug design and can also calculate the free binding energy of the bound ligands to enable prediction of their equilibrum constants. We used the AutoDock3 programme to predict the docking energies for two substituted mannosides, pNPαMan en MeUmbαMan, which are strong inhibitors of FimH-mediated adhesion. The calculated docking energies are E_{doc}=−10.4 kcal/mol for pNPαMan (K_{d}=46 nM) and E_{doc}=−10.9 kcal/mol for MeUmbαMan (K_{d}=20 nM). These computed dissociation constants are in very good agreement with the experimentally determined dissociation constants for pNPαMan (K_{d}=44 nM) and MeUmbαMan (K_{d}=20 nM). To validate the results obtained from the AutoDock3 program, the dissociation constants calculated for synthesized alkyl O-mannosides are compared with the dissociation constants determined experimentally in surface plasmon resonance measurements. Once sufficient validation is gathered, the AutoDock3 program allows many different alkyl or aryl O-mannosides and C-mannosides as well as branched O— and C-mannosides to be pre-examined for their binding to the FimH adhesin. In this way, only the predicted best binders are chemically synthesized and analysed both in vito and in vivo.

8. In vivo Anti-Adhesion Experiments

The pathogenesis of UTI has been extensively studied both in vitro and in murine and primate UTI models (Anderson et al., 2004a; Anderson et al., 2004b; Anderson et al., 2003; Bahrani-Mougeot et al., 2002; Connell et al., 1996; Hvidberg et al., 2000; Justice et al., 2004; Kau et al., 2005; Langermann and Ballou, 2003; Langermann at al., 2000; Langermann et al., 1997; Min et al., 2002; Mulvey et al., 1998; Mulvey et al., 2001; Mulvey et al., 2000; Palaszynski et al., 1998; Schilling et al., 2003a; Schilling et al., 2003b; Wu et al., 1996; Zhou et al., 2001). These studies all dearly point to the central importance of FimH-mediated adhesion for bladder infection, and also provide a strong background for design of experiments to test potential drug candidates against UTI.

Proof-of-principle that mannose derivatives of the invention can be used as anti-adhesives to eliminate/prevent UPEC bladder infection is obtained by using a murine cystitis model (see for example Anderson et al., 2003; Connell et al., 1996; Hagberg et al., 1983; Justice et al., 2004; Langermann et al., 1997; Schilling et al., 2003a; Shahin et al., 1987). Briefly, mice are inoculated with UPEC (e.g. the NU14 E. coli cystitis isolate; Hultgren et al., 1986), or with a mixture of said bacteria and a mannose derivative of the present invention, or with isogenic, non-adhesive and non-infectious bacteria (e.g. E. coli NU14-1; Langermann et al., 1997), by urethral catheterization under ether anesthesia. Bacteria in bladders (and kidneys) are quantitated by viable counts on tissue homogenates obtained at the time the mice are killed, typically 24-48 h after inoculation. Leukocyte numbers in urine samples taken at intervals after inoculation are counted using a Burker chamber (Shahin et al., 1987). Urine samples taken from individual mice before each experiment are examined for the presence of neutrophils; mice with a preexisting neutrophil response are excluded. An ability of added mannose derivatives to block infection is indicated by a reduction of viable counts/bladder and of the neutrophil count in urine.

The in vivo efficacy of mannose derivatives as anti-adhesives to eliminate/prevent UPEC bladder infection can also be tested in the cynomolgus monkey UTI model (Macaca fascicularis) (Ishikawa et al., 2004). The distribution of FimH receptors in monkey tissues has been shown to be very similar to that in humans so that urinary tract infection in the cynomolgus monkey is a relevant model of the human disease. Briefly, bladder infection is induced by inoculation of a bacterial suspension (typically 1 ml, 10⁸ cfu/ml) via urethral catheter (below the volumetric capacity of the bladder, void volume>50 ml). Bacteria used for infection are grown under conditions that maximize type 1 pilus expression (see Table 1 in Langermann et al., 2000). Infection is monitored by culture of suprapubic bladder aspiration samples. At 20-30 minutes before bladder aspiration, monkeys are hydrated with ≧50 ml of lukewarm saline administered subcutaneously for optimal diuresis. Infections persisting after ˜2.5 weeks are eliminated through intramuscular injection of a suitable antibiotic (e.g. ciproflaxin). All experiments are done under ketamine and midazolam anaesthesia. To test the ability of mannose derivatives of the present invention to block infection, mannose derivatives in suitable concentrations are mixed with UPEC (e.g. the NU14 E. coli cystitis isolate; Hultgren et al., 1986) and introduced into the monkey bladder as outlined above. UPEC without added mannose derivatives can be used as a positive control, and a Fim-negative (and therefore non-adhesive and non-infectious) E. coli strain (e.g. E. coli NU14-1; Langermann et al., 1997) can be used as negative control. The ability of mannose derivatives to prevent infection is indicated by negative cultures of bladder aspiration samples.

Materials and Methods

1. Expression and Purification of FimH

FimH truncate FimHtr_{J96} was expressed from plasmid pPKL241 (Schembri et al., 2000), FimHtr_{F18} from plasmid pPKL316, and FimHtr_{Cl#4} from plasmid pMAS146, all three coding for the lectin domain of FimH (residues 1-158) with a C-terminal 6-histidine tag. The same expression and purification protocol was used for all three variants of the protein. E. coli host strain HB101 lacking the fim operon was transformed with the FimHtr plasmid. Cells were grown in M9 minimal medium (Sambrook et al., 1989) containing 50 μg ml⁻¹ ampicillin at 37° C. At A_{600 nm}=0.6, the cells were induced with 5 mM IPTG and the cells were harvested by centrifugation 5 hours after induction. To extract the periplasm, cells were resuspended in 4 ml 20% sucrose in 20 mM Tris buffer, pH 8.0, per gram of cells. 0.2 ml 0.1 M EDTA and 40 μl lysozyme (15 mg ml⁻¹) per gram of cells were added, and the cells left to incubate on ice for 40 min. 0.16 ml of 0.5 M MgCl₂ per gram of cells were added, and the mixture centrifuged at 10000 rpm for 20 min. The supernatant, containing the periplasm, was dialysed against 300 mM NaCl, 50 mM NaPO₄ buffer, pH 7.8, over night. The protein was purified on a Pharmacia HiTrap Chelating HP 5-mI column (Pharmacia, Sweden) loaded with Ni chloride, and eluted with a sharp 0-500 mM imidazole gradient Fractions containing FimHtr were pooled, dialysed against 50 mM sodium acetate, pH 5.25, and loaded onto a Mono S HR 8-ml column. The protein was eluted with a 0-500 mM NaCl gradient, dialyzed overnight against 20 mM Tris, pH 7.5, and concentrated to about 15 mg ml⁻¹ using Vivaspin 20-ml concentrators Vivascience, UK).

2. Synthesis of Alkyl-Mannosides

Alkyl mannosides were synthesised through silver triflate-promoted couplings of the corresponding alcohol with 2,3,4,6-tetra-O-benzoyl-alpha-Dmannopyranosyl bromide, followed by Zemplén deacylation of the obtained protected alkyl mannosides, according to the procedure reported for the octyl and tetradecyl mannosides (Oscarson and Tidén, 1993).

3. Synthesis of Tri-Mannosides

Syntheses of tri-mannosides was as reported earlier by Rakesh et al., 1995, Shaheer et al., 1990 and Carole et al., 2002.

4. Binding Studies

To measure the dissociation constant for FimH:alkyl or aryl-mannoside binding, two different binding experiments were performed.

Solution Affinity Measurements at Equilibrium of FimH-Carbohydrate Interactions

Surface Plasmon Resonance measurements were performed on a Biacore3000™. The Fab fragments of a monoclonal antibody against FimH were covalently immobilised via lysines at 1000 Resonance Units (1000 pg ligand/mm²) in flowcell Fc2 on a CM5 biasensor chip (BIAapplications Handbook, Biacore AB, Uppsala, Sweden). Immobilisation buffer was 100 mM NaAc pH 5.0 with 100 mM NaCl. The reference flowcell Fc1 was left blank.

Binding of FimH to the immobilized antibody was measured with a Biacore 3000 instrument in running buffer (phosphate buffered saline with 0.005% surfactant P20 and 3 mM EDTA), on both flowcells Fc1 and Fc2 simultaneously, at a flow rate of 30 μl/min and at 25° C. Complete dissociation of FimH was done with running buffer before starting a new binding cycle. For all measurements, the association time was 3 minutes, the dissociation time was 30 min. All binding cycles were performed in duplicate, including a zero concentration cycle of FimH (injection of running buffer).

In a first experiment, the kinetic constants, kₐ and k_{d}, and the maximal binding Rₘₐₓ, were determined for the FimH-antibody interaction (FimH concentrations (nM): 2000, 1000, 500, 250, 125, 62.5, 31.25, 15.625, 7.818, 3.911, 1.957, 0). All analyses were performed with the BIAeval software. A Langmuir binding isotherm with a 1:1 stoichiometry was fitted to the data, from which the kinetic constants and maximal binding were obtained.

In the next experiment, samples containing a fixed concentration of FimH (close to the K_{D} of the FimH-antibody interaction) in combination with varying concentrations of saccharide, were used to determine the dissociation constant of the FimH-saccharide interaction in a competition experiment. First, ten-fold dilutions of the saccharide solution were used to determine the concentration range for binding of the saccharide to FimH. A Langmuir binding isotherm with a 1:1 stoichiometry was fitted to the data, using the kinetic constants and Rₘₐₓ from the first experiment, to obtain the concentrations of FimH that were free ([FimH]_{free}) to bind the antibody immobilised on the chip. Secondly, the concentration range of the saccharide was extended and adapted to assure accurate fitting, and the equilibrium binding constant of the FimH-saccharide interaction was obtained from the curve of [FimH]_{free} against concentration of saccharide. Every measurement was repeated at least twice, including testing the variation between different protein batches and where possible different saccharide stock solutions (typically 200 mM).

Displacement Assay

[³H]alpha-D-mannose was obtained from Amersham. Methyl mannoside, p-Nitrophenyl α-mannoside (pNPalpha-Man), and 4-Methylumbelliferyl α-mannoside (MeUmb-alpha-Man) were obtained from Sigma. Syntheses of tri-mannosides was as reported earlier by Oscarson and co-workers (1993). Weighed amounts of tri-mannosides were dissolved in double distilled water to give stock solutions of 0.87 M man-(1,2)-man-(1,2)-man, 0.25 M man-(1,2)-man-(1,3)-man, 0.27 M man-(1,2)-man-(1,6)-man, 0.30 M man-(1,3)-man-(1,6)-man, 0.13 M man-(1,6)-man-(1,6)-man. Similarly, alkyl mannosides were dissolved in double distilled water to give stock solutions of 100 mM methyl mannoside, 59.8 mM ethyl mannoside, 45.9 mM propyl mannoside, 51.9 mM butyl mannoside, 17.3 mM pentyl mannoside, 20.8 mM hexyl mannoside, 15.3 mM heptyl mannoside, 15.2 mM octyl mannoside. pNPalphaMan and MeUmbalphaMan (6 mg each) were dissolved in 20 μl DMSO and diluted to 20 mM using double distilled water. Binding experiments were performed using six different concentrations of [³H]-alpha-D-mannose (final concentrations 43.5 μM, 29.0 μM, 19.3 μM, 12.9 μM, 8.6 μM, 5.7 μM). FimHtr_{J96} obtained by growing bacteria in minimal medium was used in all binding experiments. 180 μl protein at a concentration of about 500 nM was mixed with 20 μl of the radioactive ligand, and incubated at 37° C. for 20 min. To separate free ligand from bound, the mixture was rapidly filtrated through a Protran BA 85 Cellulose-nitrate filter (Schleicher & Schuell, Dassel, Germany), and washed once with 1 ml of ice-cold 1× PBS (phosphate buffered saline). Filter-bound radioactivity was measured by scintillation spectrometry within 24 hours. The displacement experiments were performed using six different concentrations (final concentrations in the range 0.0˜43.5 μM) of the inhibitor, in the presence of 43.5 μM [³H]-alpha-D-mannose. 20 μl radioactive ligand, 20 μl inhibitor at decreasing concentrations, and 160 μl protein (500 nM) were mixed, and the experiments performed as above. All experiments were performed in duplicates. For determination of K_{D} for α-D-mannose, a hyperbolic curve (y=P₁x/(P₂+x), where P₂=K_{D}) was fitted to the data. For the displacement experiments, the curve y=P₁/(P₂+x), where P₂ is the concentration of the inhibitor displacing 50% of the labelled ligand, [I]_{0.5} was used instead. To calculate the inhibitor dissociation constant (K_{I}) the Cheng & Prusoff equation (K_{I}=[I]_{0.5}/([L]/K_{L}+1); K_{L} is the constant of dissociation for the ligand) (Cheng and Prusoff, 1973), was used when both the concentration of the radioactive ligand (L) and the displacing agent (I) are in excess over the protein (L_{T}>>P_{T};I_{T}>>P_{T}, T indicates total concentration). For very strong inhibitors, when I_{T} is no longer in excess over P_{T}, the equation of Homvitz et al. (K_{I}=I_{T}/((1−Y)/Y*(L_{T}/K_{L})−1)−P_{T}*K_{L}*Y/L_{T} where Y is the fraction of the ligand bound in presence of the inhibitor) (Horovitz and Levitzki, 1987) was used instead. A plot of I_{T}/((1−Y)*(L_{T}/K_{L})−Y) against 1/Y gives a straight line with a slope of K_{I}.

Tables

TABLE 1

| | K_{D} | ΔG° |
|---|---|---|
| Ligand | (nM) | (kcal/mol) |
| Mannose | 2.3 10³ | −7.6 |

linear alkyl α-D-mannosides (alkyl man)

| | | |
|---|---|---|
| Methyl man | 2.2 10³ | −7.7 |
| Ethyl man | 1.2 10³ | −8.1 |
| Propyl man | 300 | −8.9 |
| Butyl man | 151 | −9.3 |
| Pentyl man | 25 | −10.4 |
| Hexyl man | 10 | −10.9 |
| Heptyl man | 5 | −11.3 |
| Octyl man | 22 | −10.4 |

aryl α-D-mannosides (aryl man)

| | | |
|---|---|---|
| Ethylphenyl man | 86 | −9.6 |
| Ethyl aminophenyl man | 137 | −9.4 |
| p-Nitrophenyl man | 26 | −10.3 |
| Umbelliferyl man | 12 | −10.8 |

TABLE 2

| | K_{D} | ΔG° |
|---|---|---|
| Ligand | (nM) | (kcal/mol) |
| 2-deoxy α-D-mannose | 0.3 10⁶ | −4.8 |
| glucose | 9.24 10⁶ | −2.8 |
| galactose | 0.1 10⁹ | −1.4 |
| fructose | 31 μM | −6.1 |
| sucrose | 12.8 mM | −2.6 |

TABLE 3

| | K_{D} J96 | K_{D} CI#4 | K_{D} F18 | ΔG° J96 | ΔG° CI#4 | ΔG° F18 |
|---|---|---|---|---|---|---|
| Ligand | (nM) | (nM) | (nM) | (kcal/mol) | (kcal/mol) | (kcal/mol) |
| α-D-mannose | 2300 | 10700 | 9800 | −7.6 | −7.0 | −7.1 |
| man-(1,2)-man-(1,2)-man | 1600 | 3950 | 3250 | −8.2 | −7.7 | −7.8 |
| man-(1,2)-man-(1,3)-man | 1800 | 3650 | 3050 | −8.1 | −7.7 | −7.8 |
| man-(1,2)-man-(1,6)-man | 830 | 2200 | 1800 | −8.6 | −8.0 | −8.1 |
| man-(1,3)-man-(1,6)-man | 485 | 1030 | 730 | −8.6 | −8.5 | −8.7 |
| man-(1,6)-man-(1,6)-man | 1400 | 7500 | 5900 | −8.3 | −7.3 | −7.4 |
| α1-3,α1-6 mannopentaose | 330 | nd | nd | −8.8 | nd | nd |

### REFERENCES

1) Bahrani-Mougeot, F. K., Buckles, E. L., Lockatell, C. V., Hebel, J. R., Johnson, D. E., Tang, C. M., and Donnenberg, M. S. (2002) Mol Microbiol 45, 1079-1093
2) Berglund, J., and Knight, S. D. (2003) Adv Exp Med Biol 535, 33-52
3) Brinton, C. C., Jr. (1959) Nature 183, 782-786
4) Buchanan, K., Falkow, S., Hull, R. A., and Hull, S. I. (1985) J. Bacteriol 162, 799-803
5) Carole A. Bewley, Shigeki Kiyonaka and Itaru Hamachi, Journal of Molecular Biology 322 (2002) 881-889.
6) Cheng, Y., and Prusoff, W. H. (1973) Biochem Pharmacol 22, 3099-3108
7) Choudhury, D., Thompson, A., Stojanoff, V., Langermann, S., Pinkner, J., Hultgren, S. J., and Knight, S. D. (1999) Science 285, 1061-1066
8) Duchet-Suchaux M, Le Maitre C, Bertin A. (1990) J Med Microbiol. 31: 185-90.
9) Elsinghorst, E A. (1994) Measurement of invasion by gentamicin resistance. Methods Enzymol 236: 405-420.
10) Falk, P., Roth, K. A., Borén, T., Westblom, T. U., Gordon, J. I., and Normark, S. (1993) Proc Natl Acad Sci USA 90: 2035-2039
11) Firon, N., Ofek, I., and Sharon, N. (1982) Biochem Biophys Res Commun 105, 1426-1432
12) Firon, N., Ofek, I., and Sharon, N. (1983) Carbohydr Res 120, 235-249
13) Firon, N., Ofek, I., and Sharon, N. (1984) Infect Immun 43, 1088-1090
14) Foxman, B. (2002) Am J Med 113 Suppl 1A, 5S-13S
15) Gupta, K., Sahm, D. F., Mayfield, D., and Stamm, W. E. (2001) Clin Infect Dis 33, 89-94
16) Horovitz, A, and Levitzki, A. (1987) Proc Natl Aced Sci USA 84, 6654-6658
17) Hung, C. S., Bouckaert, J., Hung, D., Pinkner, J., Widberg, C., DeFusco, A., Auguste, C. G., Strouse, R., Langermann, S., Waksman, G., and Hultgren, S. J. (2002) Mol Microbiol 44, 903-915
18) Johnson, J. R., Manges, A. R., O'Bryan, T. T., and Riley, L. W. (2002) Lancet 359, 2249-2251
19) Knight, S. D., Berglund, J., and Choudhury, D. (2000) Curr Opin Chem Biol 4, 653-660
20) Langermann, S., Mollby, R., Burlein, J. E., Palaszynski, S. R., Auguste, C. G., DeFusco, A., Strouse, R., Schenerman, M. A., Hultgren, S. J., Pinkner, J. S., Winberg, J., Guldevall, L., Soderhall, M., Ishikawa, K., Normark, S., and Koenig, S. (2000) J Infect Dis 181, 774-778
21) Langermann, S., Palaszynski, S., Barnhart, M., Auguste, G., Pinkner, J. S., Burlein, J., Barren, P., Koenig, S., Leath, S., Jones, C. H., and Hultgren, S. J. (1997) Science 276, 607-611
22) Lindhorst, T. K., Kieburg, C., and Krallmann-Wenzel, U. (1998) Glycoconj J 15, 605-613
23) Martinez, J. J., Mulvey, M. A., Schilling, J. D., Pinkner, J. S., and Hultgren, S. J. (2000). EMBO J 19: 2803-2812.
24) Mulvey, M. A., Lopez-Boado, Y. S., Wilson, C. L., Roth, R., Parks, W. C., Heuser, J. & Hultgren, S. J. (1998) Science 282, 1494-1497.
25) Mulvey, M. A. (2002) Cell Microbiol 4, 257-271
26) Neeser, J.-R., Koellreutter, B., and Wuersch, P. (1986) Infect Immun 52, 428-436
27) Nicolle, L. E. (2002) J Am Genatr Soc 50, S230-235
28) O'Hanley, P., Low, D., Romero, I., Lark, D., Vosti, K., Falkow, S., and Schoolnik, G. (1985) N Engl J Med 313, 414-420
29) Oscarson S and Tidén AK (1993) Carbohydrate Research 247, 323
30) Rakesh K. Jain, Xiao-Gao Liu, Subba Rao Oruganti, E. V. Chandrasekaran, Khushi L. Matta, Carbohydrate Research 271 (1995) 185-196
31) Ronald, A. (2002) Am J Med 113, 14S-19S
32) Sambrook, J., Fritsch, E. F., and Maniatis, T. (1989) Molecular Cloning, A Laboratory Manual, 2nd Ed. (Chris Nolan, Ed.), Cold Spring Harbour Laboratory Press, New York, USA.
33) Sauer, F. G., Barnhart, M., Choudhury, D., Knight, S. D., Waksman, G., and Hultgren, S. J. (2000) Curr Opin Struct Biol 10, 548-556
34) Sauer, F. G., Futterer, K., Pinkner, J. S., Dodson, K. W., Hultgren, S. J., and Waksman, G. (1999) Science 285, 1058-1061
35) Sauer, F. G., Knight, S. D., Waksman, Gj, and Hultgren, S. J. (2000) Semin Cell Dev Biol 11, 27-34
36) Schembri, M. A., Hasman, H., and Klemm, P. (2000) FEMS Microbiol Lett 188, 147-151
37) Schilling, J. D., Mulvey, M. A., and Hultgren, S. J. (2001) J Infect Dis 183 Suppl 1, S36-40
38) Shaheer H. Khan, Rakesh K. Jain and hushi L. Matta, Carbohydrate Research 207 (1990) 57-69
39) Sokurenko, E. V., Chesnokova, V., Doyle, R. J., and Hasty, D. L. (1997) J Biol Chem 272, 17880-17886
40) Sokurenko, E. V., Chesnokova, V., Dykhuizen, D. E., Ofek, I., Wu, X. R., Krogfelt, K. A., Struve, C., Schembri, M. A., and Hasty, D. L. (1998) Proc Natl Acad Sci USA 95, 8922-8926
41) Sokurenko, E. V., Courtney, H. S., Maslow, J., Siitonen, A., and Hasty, D. L. (1995) J Bacteriol 177, 3680-3686
42) Sokurenko, E. V., Courtney, H. S., Ohman, D. E., Klemm, P., and Hasty, D. L. (1994) J Bacteriol 176, 748-755
43) Striker, R., Nilsson, U., Stonecipher, A., Magnusson, G., and Hultgren, S. J. (1995) Mol Microbiol 16: 1021-1030.
44) Thanassi, D. G., Saulino, E. T., and Hultgren, S. J. (1998) Curr Opin Microbial 1, 223-231
45) Thankavel, K., Madison, B., Ikeda, T., Malaviya, R., Shah, A. H., Arumugam, P. M., and Abraham, S. N. (1997) J Clin Invest 100, 1123-1136
46) Zafriri, D., Ofek, I., Adar, R., Pocino, M., and Sharon, N. (1989) Antimicrobial Agents And Chemotherapy 33: 92-98.
47) Zavialov, A. V., Berglund, J., Pudney, A. F., Fooks, L. J., Ibrahim, T. M., MacIntyre, S., and Knight, S. D. (2003) Cell 113, 587-596
48) Zhou, G., Mo, W. J., Sebbel, P., Min, G., Neubert, T. A., Glockshuber, R., Wu, X. R., Sun, T. T., and Kong, X. P. (2001) J Cell Sci 114, 4095-4103
49) Nguyen S. T., Grubbs R. H. and Ziller J. W., J. Am. Chem. Soc., 1993, 115, 9858.
50) Dominique R., Sas S. K., Roy R., J. Chem. Soc., Chem. Commun., 1998, 2438.
51) Sokurenko, E. V., Courtney, H. S., Maslow, J., Siitonen, A. & Hasty, D. L. (1995) J. Bacteriol. 177, 3680-3686
52) Sokurenko, E. V., McMackin, V. & Hasty, D. L. (1995) Methods Enzymol. 253, 519-528
53) Sokurenko, E. V., Chesnokova, V, Doyle, R. J. & Hasty, D. L. (1997) J. Biol. Chem. 272, 17880-17886
54) Knudsen, T. B. & Klemm, P. (1998) Microbiology 144, 1919-1929
55) Blomfield, I. C., McClain, M. S., & Eisenstein, B. I. (1991) Type 1 fimbriae mutants of Escherichia coil K12: characterization of recognized afimbriate strains and construction of new fim deletion mutants. Mol Microbiol 5: 1439-45.
56) Minion, F. C., Abraham, S. N., Beachey, E. H., & Goguen, J. D. (1989) The genetic determinant of adhesive function in type 1 fimbriae of Escherichia coli is distinct from the gene encoding the fimbrial subunit J Bacteriol 165:1033-36.
57) Furste, J. P., Pansegrau, W., Frank, R., Blocker, H., Scholz, P., Bagdasarian, M., & Lanka, E. (1986) Molecular cloning of the plasmid RP4 primase region in a multi-host-range tacP expression vector. Gene 48: 119-31.
58) Falk, P., Boren, T., Haslam, D., & Caparon, M. (1994) Bacterial Adhesion and Colonization Assays. Methods in Cell Biology, Vol 45 45: 165-192.
59) Morris, G. M., Goodsell, D. S., Halliday, R. S., Huey, R., Hart, W. E., Belew, R. K., & Olson, A J. (1998) Automated docking using a Lamarckian genetic algorithm and an empirical binding free energy function. J. Comput Chem 19: 1639-1662.
60) Anderson, G. G., Dodson, K. W., Hooton, T. M. and Hultgren, S. J. (2004a) Intracellular bacterial communities of uropathogenic Escherichia coli in urinary tract pathogenesis. Trends Microbiol, 12, 424-430.
61) Anderson, G. G., Martin, S. M. and Hultgren, S. J. (2004b) Host subversion by formation of intracellular bacterial communities in the urinary tract. Microbes Infect, 6, 1094-1101.
62) Anderson, G. G., Palermo, J. J., Schilling, J. D., Roth, R., Heuser, J. and Hultgren, S. J. (2003) Intracellular bacterial biofilm-like pods in urinary tract infections. Science, 301, 105-107.
63) Bahrani-Mougeot, F. K., Buckles, E. L., Lockatell, C. V., Hebel, J. R., Johnson, D. E., Tang, C. M. and Donnenberg, M. S. (2002) Type 1 fimbriae and extracellular polysaccharides are preeminent uropathogenic Escherichia coli virulence determinants in the murine urinary tract. Mol Microbiol, 45, 1079-1093.
64) Connell, I., Agace, W., Klemm, P., Schembri, M., Marild, S. and Svanborg, C. (1996) Type 1 fimbrial expression enhances Escherichia coli virulence for the urinary tract. Proc Natl Acad Sci USA, 93, 9827-9832.
65) Hagberg, L., Engberg, I., Freter, R., Lam, J., Oiling, S. and Svanborg Eden, C. (1983) Ascending, unobstructed urinary tract infection in mice caused by pyelonephritogenic Escherichia coli of human origin. Infect Immun, 40, 273-283.
66) Hultgren, S. J., Schwan, W. R., Schaeffer, A. J. and Duncan, J. L. (1986) Regulation of production of type 1 pill among urinary tract isolates of Escherichia coil. Infect Immun, 54, 613-620.
67) Hvidberg, H., Struve, C., Krogfelt, K. A., Christensen, N., Rasmussen, S. N. and Frimodt-Moller, N. (2000) Development of a long-term ascending urinary tract infection mouse model for antibiotic treatment studies. Antimicrob Agents Chemother, 44, 156-163.
68) Ishikawa, K., Sundelin, B., Moliby, R., Normark, S. and Winberg, J. (2004) Receptors for Escherichia coli adhesins in the genitourinary tract in a non-human primate. Scand J Urol Nephrol, 38, 3-14.
69) Justice, S. S., Hung, C., Theriot, J. A., Fletcher, D. A., Anderson, G. G., Footer, M. J. and Hultgren, S. J. (2004) Differentiation and developmental pathways of uropathogenic Escherichia coli in urinary tract pathogenesis. Proc Natl Acad Sci USA, 101, 1333-1338.
70) Kau, A. L., Hunstad, D. A. and Huitgren, S. J. (2005) Interaction of uropathogenic Escherichia coli with host uroepithelium. Curr Opin Microbiol, 8, 54-59.
71) Langermann, S. and Ballou, W. R. (2003) Development of a recombinant FimCH vaccine for urinary tract infections. Adv Exp Med Biol, 539B, 635-653.
72) Langermann, S., Mollby, R., Burlein, J. E., Palaszynski, S. R., Auguste, C. G., DeFusco, A., Strouse, R., Schenerman, M. A., Hultgren, S. J., Pinkner, J. S., Winberg, J., Guldevall, L., Soderhall, M., Ishikawa, K., Normark, S. and Koenig, S. (2000) Vaccination with FimH adhesin protects cynomolgus monkeys from colonization and infection by uropathogenic Escherichia coil. J Infect Dis, 181, 774-778.
73) Langermann, S., Palaszynski, S., Barnhart, M., Auguste, G., Pinkner, J. S., Burlein, J., Barren, P., Koenig, S., Leath, S., Jones, C. H. and Hultgren, S. J. (1997) Prevention of mucosal Escherichia coli infection by FimH-adhesin-based systemic vaccination. Science, 276, 607-611.
74) Min, G., Stolz, M., Zhou, G., Liang, F., Sebbel, P., Stoffler, D., Glockshuber, R., Sun, T. T., Aebi, U. and Kong, X. P. (2002) Localization of uroplakin la, the urothelial receptor for bacterial adhesin FimH, on the six inner domains of the 16 nm urothelial plaque partide. J Mol Biol, 317, 697-706.
75) Mulvey, M. A., Lopez-Boado, Y. S., Wilson, C. L., Roth, R., Parks, W. C., Heuser, J. and Hultgren, S. J. (1998) Induction and evasion of host defenses by type 1-piliated uropathogenic Escherichia coli. Science, 282, 1494-1497.
76) Mulvey, M. A., Schilling, J. D. and Hultgren, S. J. (2001) Establishment of a persistent Escherichia coli reservoir during the acute phase of a bladder infection. Infect Immun, 69, 4572-4579.
77) Mulvey, M. A., Schilling, J. D., Martinez, J. J. and Hultgren, S. J. (2000) Bad bugs and beleaguered bladders: interplay between umpathogenic Escherichia coli and innate host defenses. Proc Natl Acad Sci USA, 97, 8829-8835.
78) Palaszynski, S., Pinkner, J., Leath, S., Barren, P., Auguste, C. G., Burlein, J., Hultgren, S. and Langernann, S. (1998) Systemic immunization with conserved pilus-associated adhesins protects against mucosal infections. Dev Biol Stand, 92, 117-122.
79) Schilling, J. D., Martin, S. M., Hung, C. S., Lorenz, R. G. and Hultgren, S. J. (2003a) Toll-like receptor 4 on stromal and hematopoietic cells mediates innate resistance to uropathogenic Escherichia coli. Proc Natl Acad Sci USA, 100, 4203-4208.
80) Schilling, J. D., Martin, S. M., Hunstad, D. A., Patel, K. P., Mulvey, M. A., Justice, S. S., Lorenz, R. G. and Hultgren, S. J. (2003b) CD14- and Toll-like receptor-dependent activation of bladder epithelial cells by lipopolysaccharide and type 1 piliated Escherichia coli. Infect Immun, 71, 1470-1480.
81) Shahin, R. D., Engberg, I., Hagberg, L. and Svanborg Eden, C. (1987) Neutrophil recruitment and bacterial dearance correlated with LPS responsiveness in local gram-negative infection. J Immunol, 138, 3475-3480.
82) Wu, X. R., Sun, T. T. and Medina, J. J. (1996) In vitro binding of type 1-fimbriated Eschenchia coli to uroplakins 1a and 1b: Relation to urinary tract infections. Proc Natl Acad Sci USA, 93, 9630-9635.
83) Zhou, G., Mo, W. J., Sebbel, P., Min, G., Neubert, T. A., Glockshuber, R., Wu, X. R., Sun, T. T. and Kong, X. P. (2001) Uroplakin Ia is the urothelial receptor for uropathogenic Escherichia coli: evidence from in vitro FimH binding. J Cell Sci, 114, 4095-4103.

1-9. (canceled)

10. A method of treating a subject suffering from an infection of a Gram-negative bacterium, said method comprising:
providing a subject suffering from an infection of a Gram-negative bacterium with a composition comprising: 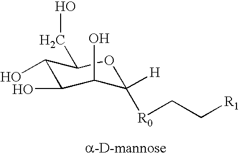
wherein R₀═O, CH₂ or S
and
—R₁═—CH₂CH₃ (ethyl), or
—CH₂CH₂CH₃ (n-propyl), or
—CH₂CH₂CH₂COOH (3-carboxypropyl), or
—CH₂CH₂CH₂CHO (4-oxobutyl), or
—CH₂CH₂CH₂CH₃ (n-butyl), or
—CH₂CH₂CH₂CF₃ (4,4,4-trifluorobutyl), or
—CH₂CH₂CH₂CH₂OH (4-hydroxybutyl), or
—CH₂CH₂CH₂CH₂CHO (5-oxopentyl), or
—CH₂CH₂CH₂CH₂CH₃ (n-pentyl), or
—CH₂CH₂CH₂CH₂CF₃ (5,5,5-trifluoropentyl), or
—CH₂CH₂CH₂CH₂COOH (4-carboxybutyl), or
—CH₂CH₂CH₂CH₂NH₂ (4-aminobutyl), or
—C₆H₁₁OH (4-hydroxycyclohexyl), or
—C₆H₁₁CF₃ (4-trifluoromethylcyclohexyl), or
—C₆H₅ (phenyl), or
—C₆H₄OH (p-hydroxyphenyl), or
—C₆H₄NH₂ (p-aminophenyl), or
—C₆H₄NO₂ (p-nitrophenyl), or
—C₆H₄COOH (p-carboxyphenyl), or
—C₆H₄CH₃ (p-methylphenyl), or
—C₆H₄CF₃ (p-trifluoromethylphenyl), or
—C₆H₄CHO (p-formylphenyl), or
—C₄H₅N₂ (pyrimidyl), or
—C₄H₄N₂OH (2-hydroxypyrimidyl), or
—C₆H₁₁ (cyclohexyl)

11. The method according to claim 10, wherein said Gram-negative bacterium comprises a type-1 pilus.

12. The method according to claim 10, wherein said Gram-negative bacterium is selected from the group consisting of Klebsiella pneumoniae, Haemophilus influenza, Shigella species, Salmonella typhimurium, Bordetella pertussis, Yersinia enterolytica, Helicobacter pylor, Proteus species and Escherichia coli.

13. The method according to claim 10, wherein said infection is a urinary tract infection.

14. The method according to claim 13, wherein said urinary tract infection is caused by E. coli.

15. The method according to claim 10, wherein said infection is a gastrointestinal infection.

16. The method according to claim 15, wherein said gastrointestinal infection is caused by Escherichia, Salmonella, Shigella and/or Yersinia species.

17. The method according to claim 10, wherein said infection is a pulmonary infection.

18. The method according to claim 17, wherein said pulmonary infection is caused by Haemophilus influenzae, Bordetella pertussis and/or Klebsiella species.
